(19) United States
(12) Patent Application Publication
(10) Pub. No.: US 2020/0015642 A1
(43) Pub. Date: Jan. 16, 2020

(54) SANITARY TOILET SYSTEMS

(71) Applicant: Shujen Yu, Henderson, NV (US)

(72) Inventor: Shujen Yu, Henderson, NV (US)

(21) Appl. No.: 16/032,560

(22) Filed: Jul. 11, 2018

(51) Int. Cl. A47K 17/00; A61L 2/10; A61L 2/24

(52) U.S. Cl. CPC A47K 17/00; A61L 2202/14; A61L 2/24; A61L 2/10

(57) ABSTRACT

Systems and methods for irradiating sanitary toilets are disclosed. According to one embodiment of the disclosure, an irradiation system may include one or more ultraviolet lights attached to one of: an interior perimeter of a toilet bowl, an underside of an inwardly facing edge of a toilet seating surface, or an inwardly facing surface of a urinal. The system may also include a controller connected to a power source and configured to activate the one or more ultraviolet lights.

### BACKGROUND

Toilets and urinals may foster germs, bacteria and/or viruses. When people are exposed to germs, bacteria, and viruses that may reside in toilets and urinals, they may be prone to a plethora of diseases, such as, for example, urinary tract infections, pneumonia, skin rashes, and so on. Flushing a toilet or urinal exacerbates the problem by allowing a plume containing germs, bacteria and/or viruses to rise and spread throughout the surrounding area.

### SUMMARY

Some or all of the above needs and/or problems may be addressed by certain embodiments of the disclosure. Systems and methods for irradiation of sanitary toilets and urinals are disclosed in accordance with example embodiments of the disclosure. According to one embodiment of the disclosure, an irradiation system may be disclosed. The irradiation system may include one or more ultraviolet lights attached to one of: an interior perimeter of a toilet bowl, an underside of an inwardly facing edge of a toilet seating surface, or an inwardly facing surface of a urinal. The irradiation system may further include a controller connected to a power source and configured to activate the one or more ultraviolet lights.

According to another embodiment of the disclosure, a method of irradiating a sanitary system may be disclosed. The method may include receiving an input from one or more sensors indicative of at least one of: a user proximity, a flushing of water into a toilet bowl, a flushing of water into a urinal, or a user activation. The method may further include activating one or more ultraviolet lights based at least in part on the received input from one or more sensors. The one or more ultraviolet lights may be attached to one of: an interior perimeter of the toilet bowl, an underside of an inwardly facing edge of a toilet seating surface, or an inwardly facing surface of the urinal.

According to another embodiment of the disclosure, a system may be disclosed. The system may include a processor and a memory comprising computer-executable instructions that may be capable of receiving an input from one or more sensors indicative of at least one of: a user proximity, a flushing of water into a toilet bowl, a flushing of water into a urinal, or a user activation. The computer-executable instructions that may be further capable of activating one or more ultraviolet lights based at least in part on the received input from one or more sensors. The one or more ultraviolet lights may be attached to one of: an interior perimeter of the toilet bowl, an underside of an inwardly facing edge of a toilet seating surface, or an inwardly facing surface of the urinal.

Other embodiments, features, and aspects of the disclosure will become apparent from the following description taken in conjunction with the following drawings.

### BRIEF DESCRIPTION OF THE DRAWINGS

The detailed description is described with reference to the accompanying figures. The use of the same reference numbers in different instances in the description and the figures may indicate similar or identical items. Various embodiments or examples (“examples”) of the present disclosure are disclosed in the following detailed description and the accompanying drawings. The drawings are not necessarily to scale. In general, operations of disclosed processes may be performed in an arbitrary order, unless otherwise provided in the claims.

FIG. 1 illustrates an example toilet system 100 with bacterial and viral dispersion immediately after flushing;

FIG. 2 illustrates an example table 200 of various bacterial and viral microorganisms and associated possible diseases;

FIG. 3 illustrates an example plume dispersion 300 at various times after a toilet has been flushed, in accordance with an example embodiment of the present disclosure;

FIG. 4 is a plan view of an irradiation system 400 installed in a toilet bowl, in accordance with an example embodiment of the present disclosure;

FIG. 5 is a cross-sectional view 5-5 of an irradiation system 400 installed in a toilet bowl, in accordance with an example embodiment of the present disclosure;

FIG. 6 is top view of an irradiation system 600 installed in a toilet bowl in accordance with an example embodiment of the present disclosure;

FIG. 7 is a perspective view an irradiation system 700 installed in a toilet seating surface 705 in accordance with an example embodiment of the present disclosure;

FIG. 8 is a perspective view an irradiation system 800 installed in a toilet seating surface 805 in accordance with an example embodiment of the present disclosure.

FIG. 9A and FIG. 9B show perspective and cross-sectional views view an irradiation system 900 installed in a toilet seating surface 705, wherein the toilet seat 705 is installed on a toilet, in accordance with an example embodiment of the present disclosure.

FIG. 10 is a side view of a view an irradiation system 1000 installed in a toilet seating surface 1005, wherein the toilet seat 1005 is installed on a toilet, in accordance with an example embodiment of the present disclosure;

FIG. 11 is a perspective view of an irradiation system 1100 installed in a toilet seating surface 1105, wherein the toilet seat 1105 is installed on a toilet, in accordance with an example embodiment of the present disclosure;

FIG. 12A and FIG. 12B show perspective views of an irradiation system 1200 installed in a urinal 1205 in accordance with an example embodiment of the present disclosure;

FIG. 13A and FIG. 13B show perspective and cross-sectional views of an irradiation system 1300 installed in a urinal 1305 in accordance with an example embodiment of the present disclosure;

FIG. 14 illustrates an example control system configured for providing systems and methods for an irradiation system for sanitary systems in accordance with certain embodiments of the disclosure; and

FIG. 15 illustrates an example flowchart of a process for irradiation of sanitary systems in accordance with certain embodiments of the disclosure.

### DETAILED DESCRIPTION

### Overview

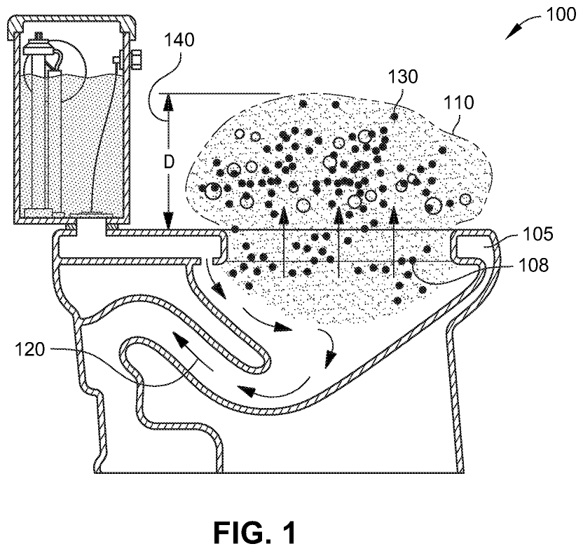

The following text sets forth a detailed description of numerous different embodiments of the disclosure. Toilets and urinals are part everyone's daily routine. When a typical toilet or urinal is used or flushed, an invisible mist or plume may be ejected from a bowl of the toilet or urinal that may extend from an opening in the bowl. FIG. 1 shows an example toilet system 100 with a plume 110 containing germs, bacteria, and viruses 130 immediately after the toilet bowl 105 has been flushed with flush water 120. As indicated in FIG. 1, the plume may extend from the opening 108 of the toilet bowl 105 up to a distance d 140. The distance d 140 and the shape of the plume 110 may depend on the size of the opening 108, the rate of flushing, the shape of the toilet bowl 105, and so on. While a toilet system 100 is illustrated in FIG. 1, it may be appreciated that a plume similar to plume 110 containing germs, bacteria, and viruses 130 may arise in a urinal or other type of sanitary systems. It may also be appreciated that the shape and size of the toilet system 100 indicated in FIG. 1 is for illustrative purposes only, and that the toilet system 100, the urinal or other sanitary systems may be of different shapes and sizes.

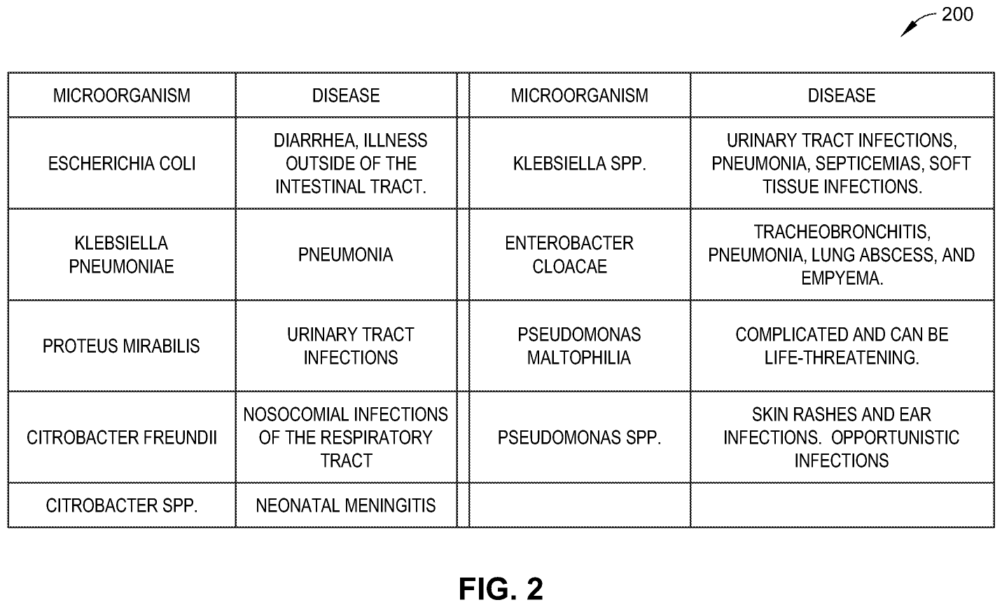

Referring now to FIG. 2, an example table 200 illustrates various microorganisms that may be part of the germs and bacteria 130 in the plume 110 that may extend from the opening 108 of the toilet bowl 105 and corresponding diseases that they may cause in a person if infected by the microorganism. By way of an example, if the plume 110 contains Escherichia coli, and it infects a user of the toilet system 100, it may cause diarrhea or illness outside of the intestinal tract of the user.

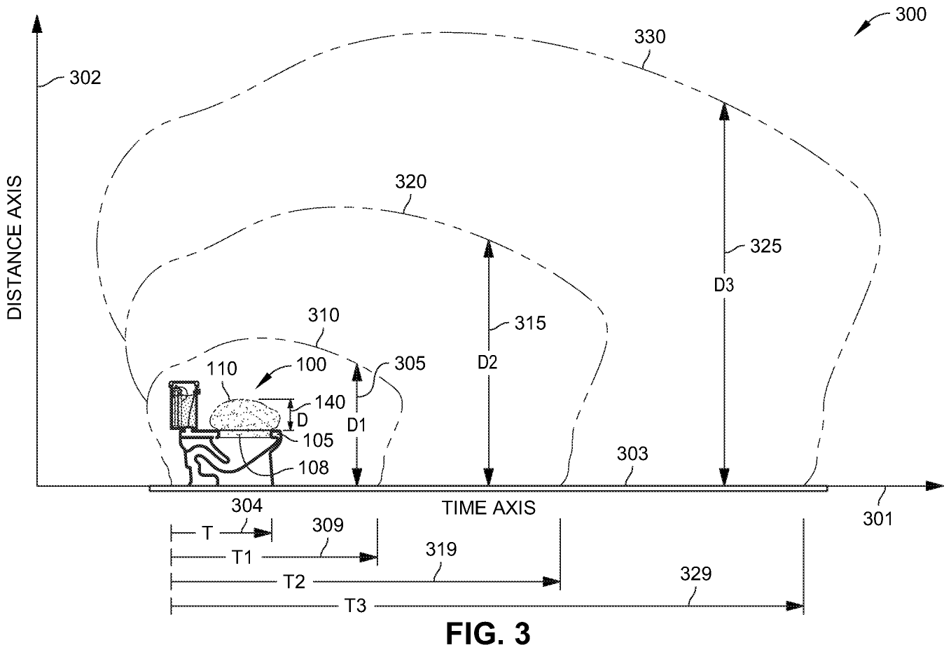

The invisible mist or plume 110 indicated in FIG. 1 may slowly disperse and balloon up over time after the toilet system 100 has been flushed. This mist may contaminate items around the toilet or urinal such as towels, walls, floor, sanitary items, and so on. The mist may be filled with germs and bacteria and may cause a health hazard for users of the toilets and urinals. Closing the lid of toilets may not eliminate the germ and/or bacteria filled mist from escaping, due to gaps between the lid, the toilet seat and the rim of the toilet bowl. FIG. 3 illustrates an example plume dispersion 300 as a function of time. FIG. 3 may be illustrative of plume propagation and impact of germs and microorganisms far removed from the toilet system 100. The example toilet system 100 is illustrated along with Time 301 on the x-axis and distance 302 from a reference floor 303 along the y-axis. As explained in reference to FIG. 1, a plume 110 containing germs, bacteria and viruses 130 is generated immediately after the toilet system 100 is flushed. This may be indicated by time T 304 and a distance d 140. In an example embodiment of the disclosure, the time T 304 may be of an order of 0-1 minutes after the toilet system 100 has been flushed. After a time T1 309 has elapsed the plume may be dispersed to a location 310 where the plume may be at a distance d1 305 from the reference floor 303. The distance d1 305 may be of the order of 2 ft from the reference floor and the time T1 309 may be of the order of 30 minutes from the time the toilet system 100 was flushed. After a time T2 319, the plume may be at location 320 with a distance d2 315 from the reference floor 303. In this case, the time T2 319 may be of the order of 60 minutes after flushing of the toilet system 100 and the distance d2 315 may be of the order of 4 feet from the reference floor 303. The example plume dispersion 300 may further be located at location 330 after a time T3 329 after flushing of the toilet system 100 at a distance d3 325 from the reference floor. As an example, the time T3 329 may be of an order of 120 minutes after flushing the toilet system 100 and the distance d3 325 may be of an order of 6 feet from the reference floor 303. For times greater than T3 329, the entire area around the toilet system 100 may be filled with the plume, causing objects located far away from the toilet system 100 to be contaminated with germs, bacteria and viruses 130.

An example embodiment of the disclosure enables irradiating the toilet system 100 such that the germs, bacteria and viruses 130 of the plume of FIGS. 1 and 3 may be eliminated. A technical effect of irradiating the toilet system 100 may be to reduce illnesses and provide a healthier surrounding near any sanitary toilet system, such as the toilet system 100 of FIG. 1. Another technical effect for a user of the toilet system may be increased confidence and peace of mind in using the toilet system 100.

### Example Implementations

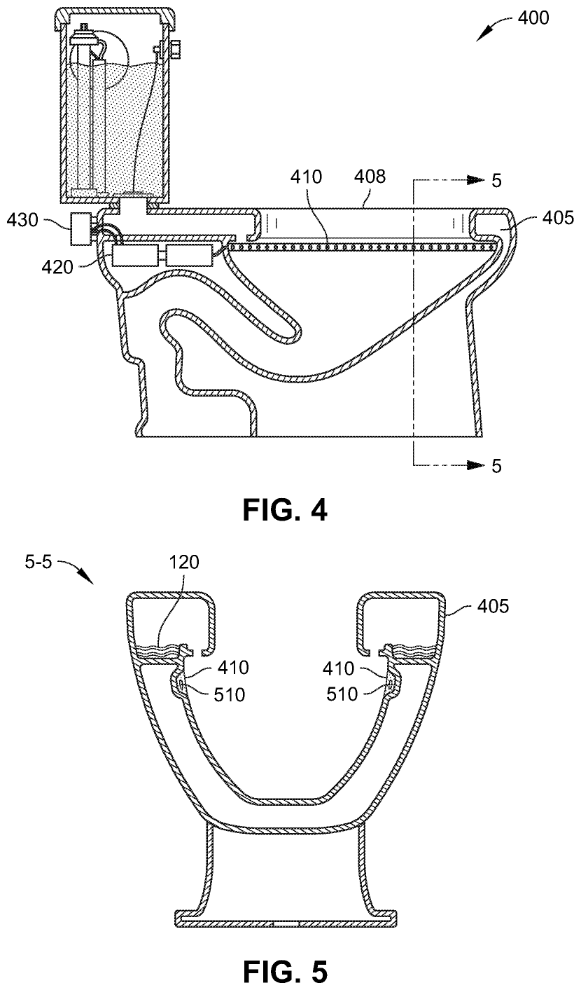

Embodiments of the disclosure may be described in the attached figures, FIG. 4 through FIG. 15. Referring now to FIG. 4, embodiments of the disclosure may be used in conjunction within an environment of an irradiation system 400. System 400 may include a sanitary system, such as a toilet bowl 405 as indicated in FIG. 4 or it may include a urinal (not shown in FIG. 4). As illustrated in FIG. 4, one or more ultraviolet lights 410 may be attached to an interior perimeter of a toilet bowl 405. The system 400 may include a controller 420 connected to power source 430. The controller 420 may be configured to activate the one or more ultraviolet lights 410. In an example embodiment, the one or more ultraviolet lights 410 may be ultraviolet-C lights. In other example embodiments, the one or more ultraviolet lights 410 may be ultraviolet-A lights or ultraviolet-A lights. The one or more ultraviolet lights may be attached to the toilet bowl 405 just below a rim 406 in the interior perimeter of the toilet bowl 405.

Referring again to FIG. 4, the control system or controller 420 may be configured to activate the one or more ultraviolet lights 410 based at least in part on an input from one or more sensors (not shown) indicative of at least one of: a user proximity, an indication of impending flushing of water into the toilet bowl 405 or a user activation. By way of an example, a motion sensor (not shown) may indicate proximity of a user to the toilet bowl 405, and this may be communicated to the controller 420, that may in turn trigger activation of the one of more ultraviolet lights 410. In another example embodiment of the disclosure, a sensor may indicate impending flushing of the toilet (not shown), which may then trigger the controller 420 to activate the one or more ultraviolet lights 410. The sensor may indicate impending flushing of the toilet bowl 405 by measuring water flow from the water tank 435 or based on a timer or based on a motion sensor detecting a user's movements. It must be appreciated that the controller 420 may be a separate entity or it may be integrated into the toilet bowl system 405. In another example embodiment, an automatic flushing system may be disclosed, that may be integrated into the toilet bowl 405.

FIG. 5 illustrates a cross section 5-5 of the toilet bowl 405 of the irradiation system 400. Also indicated are the one or more ultraviolet lights 410 that may be attached to the toilet bowl 405. In example embodiment of the disclosure, the one or more ultraviolet lights 410 may be attached just below the rim 406 of the toilet bowl 405 via a water insoluble resin 510. It is to be understood that the one or more ultraviolet lights 410 may be attached to the interior perimeter of the toilet bowl 405 using a variety of techniques. Also, the pattern and configuration of the one or more ultraviolet lights 410 indicated in FIGS. 4 and 5 is for illustrative purpose only. The ultraviolet lights 410 may be disposed on the toilet bowl in a variety of configurations.

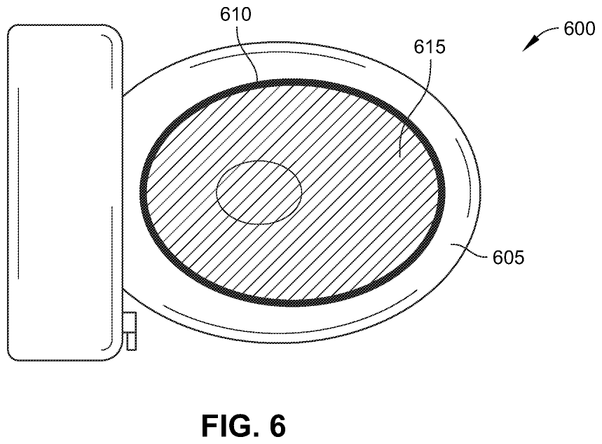

Referring now to FIG. 6, in an example embodiment of the disclosure, a toilet system 600 with example ultraviolet lights 610 is illustrated. In this example embodiment, the ultraviolet lights completely cover an area 615 formed by the toilet bowl 605, so as to irradiate the entire region of area 615. It may be appreciated in FIGS. 4, 5, and 6 that the one or more ultraviolet lights 410 or 610 may remain activated for a period of time after the flushing of water into the toilet bowl 405 or 605. The period of time after the flushing of water into the toilet bowl 405 or 605 may depend on the size of the toilet bowl 405 or 605, the type of excrement being flushed, and so on and so forth. In certain example embodiments of the disclosure, the period of time after the flushing of the toilet bowl 405 or 605 may be set as 1 minute, 2 minutes, or 5 minutes.

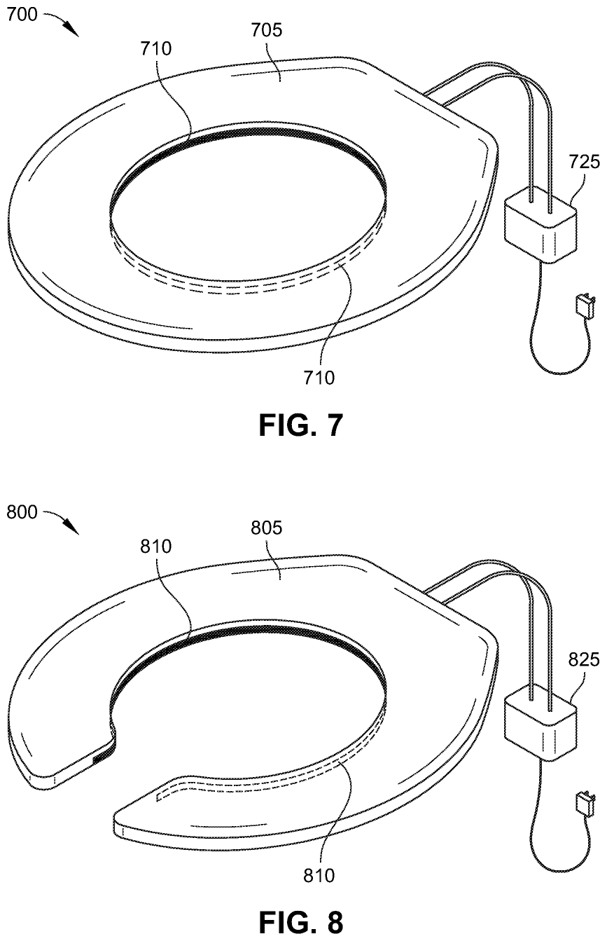

Referring now to FIG. 7, embodiments of the disclosure may be used in conjunction within an environment of an irradiation system 700. System 700 may include a sanitary system, such as a toilet seating surface 705 as indicated in FIG. 7. As illustrated in FIG. 7, one or more ultraviolet lights 710 may be attached to an underside of an inwardly facing edge of a toilet seating surface 705. The system 700 may include a controller coupled to a power source in a single unit 725. The controller and power supply 725 may be configured to activate the one or more ultraviolet lights 710. In an example embodiment, the one or more ultraviolet lights 710 may be ultraviolet-C lights. In other example embodiments, the one or more ultraviolet lights 710 may be ultraviolet-A lights or ultraviolet-A lights. The one or more ultraviolet lights may be attached to the toilet seating surface 705 just below an edge of the toilet seating surface 705.

Referring again to FIG. 7, the control system or controller and power supply 725 may be configured to activate the one or more ultraviolet lights 710 based at least in part on an input from one or more sensors (not shown) indicative of at least one of: a user proximity, an impending flushing of water into the toilet bowl (not shown) or by a user activation. By way of an example, a motion sensor (not shown) may indicate proximity of a user to the toilet seating surface 705, and this may be communicated to the controller and power supply 725, that may in turn trigger activation of the one of more ultraviolet lights 710. In another example embodiment of the disclosure, a sensor may indicate an impending flushing of the toilet (not shown), which may then trigger the controller 725 to activate the one or more ultraviolet lights 710. The sensor may indicate an impending flushing of the toilet bowl (not shown) by measuring water flow from a water tank (not shown), based on a timer or based on a motion sensor detecting a user's movements.

FIG. 8 illustrates an example embodiment of a irradiation system 800 that may be configured in a toilet seating surface 805. One or more ultraviolet lights 810 may be disposed along the inner and outer surfaces of the toilet seating surfaces and may be activated when a controller 825 receives a signal from one or more sensors indicating proximity of a user or flushing of a toilet.

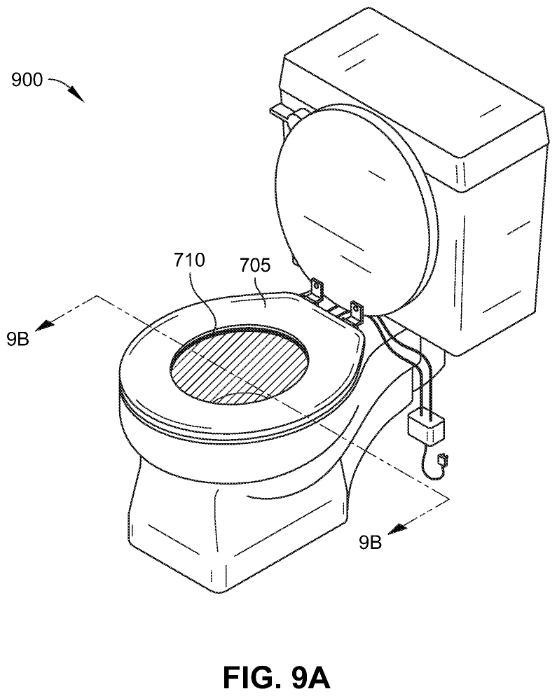

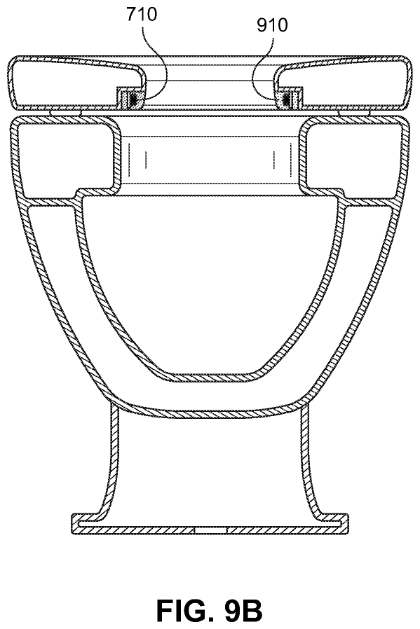

Referring now to FIGS. 9A and 9B, in an example embodiment of the disclosure, perspective and cross-sectional views respectively of an irradiation system 900 with one or more ultraviolet lights 710 along a toilet seating surface 705 is illustrated. FIG. 9B illustrates an exploded view of cross-section 9B-9B of FIG. 9A. Also indicated in FIG. 9B is a water insoluble resin 910 that may be used to attach the one or more ultraviolet lights 710 to the toilet seating surface 705.

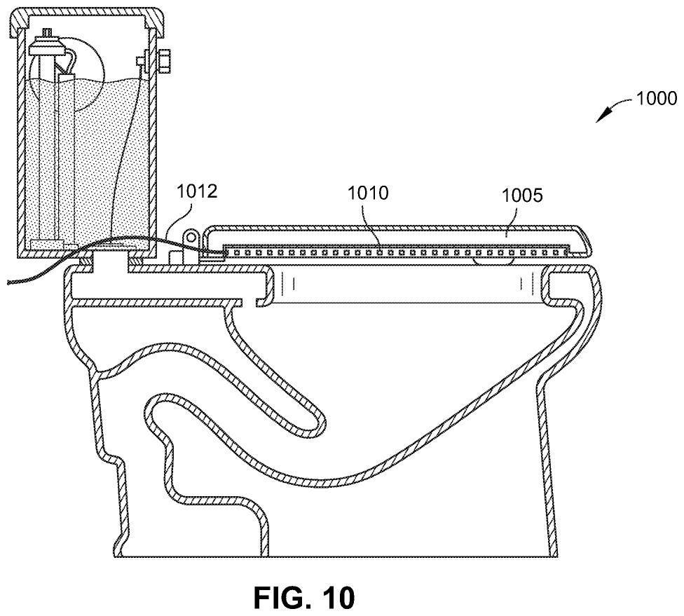

FIG. 10 illustrates a side view of a irradiation system 1000, wherein one or more ultraviolet lights 1010 installed along an underside of a toilet seating surface 1005 is indicated. Also shown is a possible power supply arrangement 1012 for the one or more ultraviolet lights 1010.

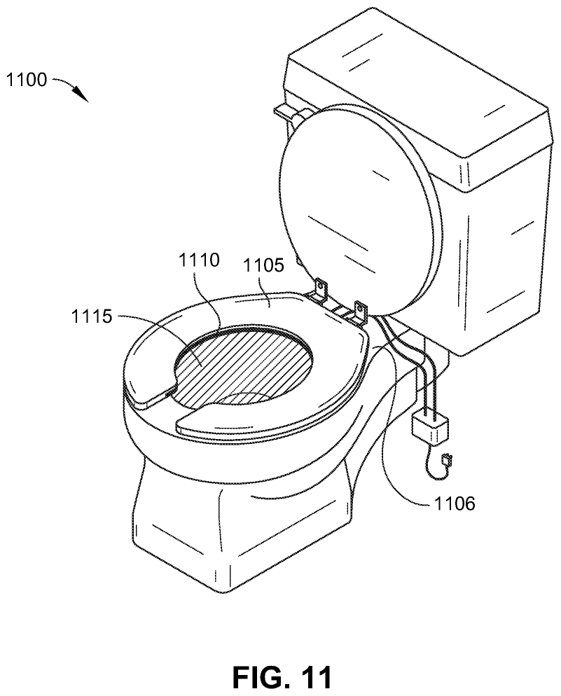

FIG. 11 illustrates a perspective view of an irradiation system 1100 with one or more ultraviolet lights 1110 mounted to the lower part of the inside perimeter of a toilet seating surface 1105. In this example embodiment, the ultraviolet lights completely cover an area 1115 formed by the toilet bowl 1106, so as to irradiate the entire region of area 1115. It may be appreciated in FIGS. 7-11 that the one or more ultraviolet lights 710, 810, 910, 1010, or 1110 may be activated based on a proximity of a user, for example, when a user is detected, and remain activated for a period of time during usage of the toilet system, and before, during, and after the flushing of water into the toilet bowl associated with the toilet seating surface 705, 805, 905, 1005, or 1105. As described earlier, the period of time after the flushing of water into the toilet bowl may depend on the size of the toilet bowl, the type of excrement being flushed, and so on and so forth. In certain example embodiments of the disclosure, the period of time after the flushing of the toilet bowl may be set as 1 minute, 2 minutes, or 5 minutes.

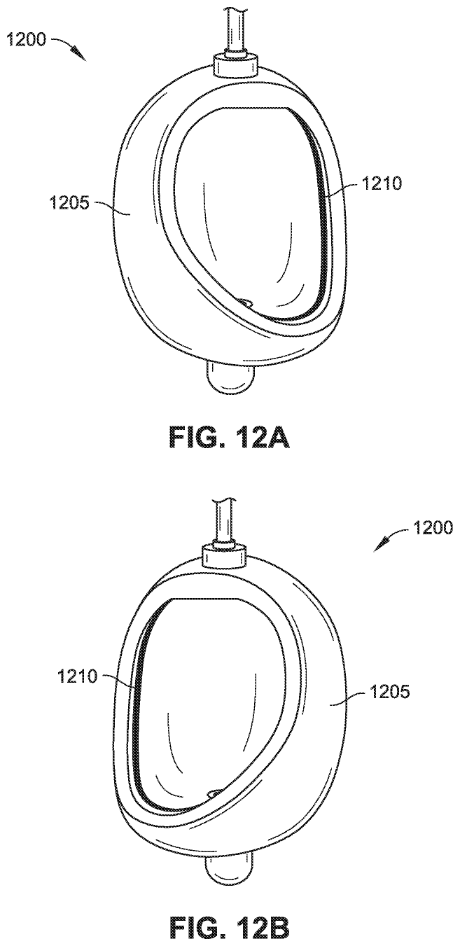

Attention is now drawn to FIGS. 12A and 12B, which illustrate perspective views of an irradiation system 1200 that may be installed in a urinal 1205 in accordance with an embodiment of the disclosure. As indicated in FIG. 12A and FIG. 12B, the one or more ultraviolet lights may be attached to an inwardly facing surface of a urinal 1205. The system 1200 may include a controller (not shown) connected to power source (not shown). The controller may be configured to activate the one or more ultraviolet lights 1210. In an example embodiment, the one or more ultraviolet lights 1210 may be ultraviolet-C lights. In other example embodiments, the one or more ultraviolet lights 1210 may be ultraviolet-A lights or ultraviolet-A lights. The one or more ultraviolet lights may be attached to the urinal 1205 just below inside a rim in the interior perimeter of the urinal 1205.

Referring again to FIG. 12A and FIG. 12B, the control system or controller may be configured to activate the one or more ultraviolet lights 1210 based at least in part on an input from one or more sensors (not shown) indicative of at least one of: a user proximity, a flushing of water into the urinal 1205 or a user activation. By way of an example, a motion sensor (not shown) may indicate proximity of a user to the urinal 1205, and this may be communicated to the controller, that may in turn trigger activation of the one of more ultraviolet lights 1210. In another example embodiment of the disclosure, a sensor may indicate flushing of the urinal (not shown), which may then trigger the controller to activate the one or more ultraviolet lights 1210. The sensor may indicate flushing of the urinal 1205 by measuring water flow from a water tank (not shown).

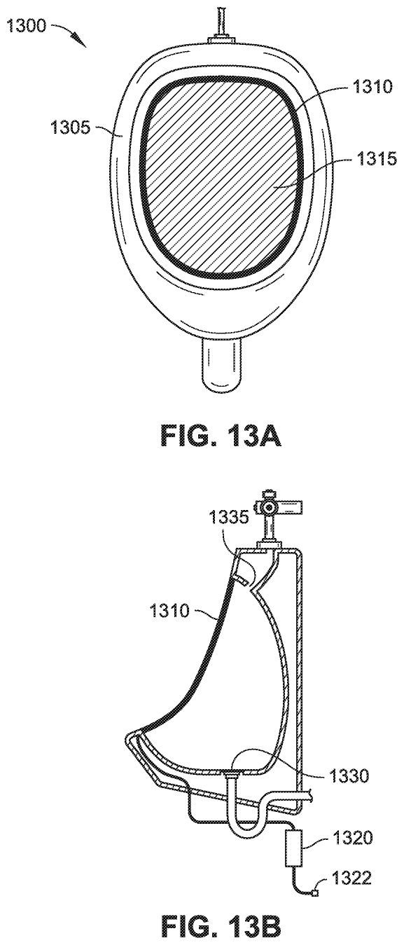

Referring now to FIG. 13A and FIG. 13B, in an example embodiment of the disclosure, respective perspective and cross-sectional views of an irradiation system 1300 installed in a urinal 1305 along with example ultraviolet lights 1310 is illustrated. In this example embodiment, the ultraviolet lights completely cover an area 1315 formed by the urinal 1305, so as to irradiate the entire region of area 1315. Also indicated in FIGS. 13A and 13B are a controller 1320 connected to a power source 1322, a drain 1330, and a flushing outlet 1335. It may be appreciated in FIGS. 12 and 13 that the one or more ultraviolet lights 1210 or 1310 may remain activated for a period of time after the flushing of water into the urinal 1205 or 1305. The period of time after the flushing of water into the urinal 1205 or 1305 may depend on the size of the urinal 1205 or 1305, the type of excrement being flushed, and so on and so forth. In certain example embodiments of the disclosure, the period of time after the flushing of the urinal 1205 or 1305 may be set as 1 minute, 2 minutes, or 5 minutes.

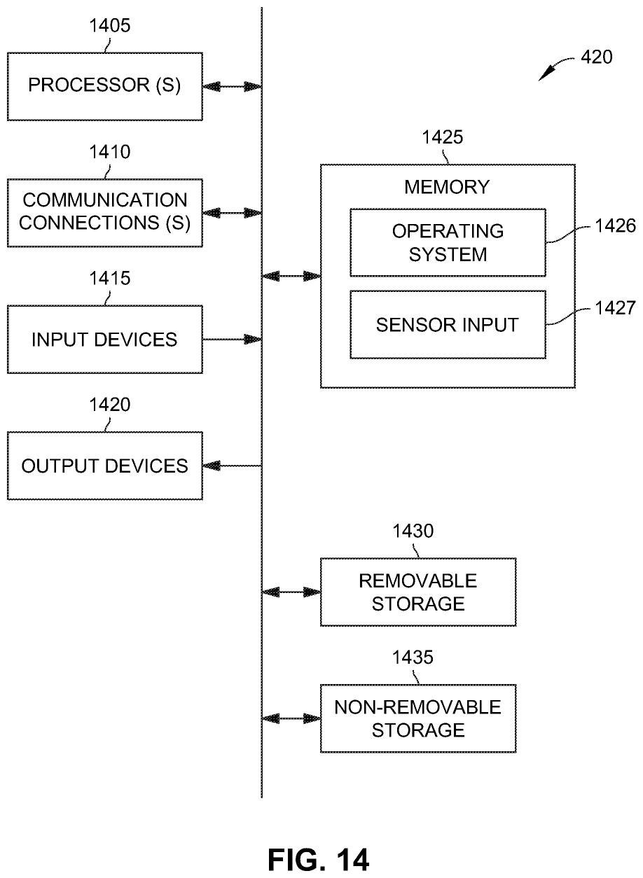

Attention is now drawn to FIG. 14, which illustrates an example controller 420 configured for implementing certain systems and methods for irradiation of sanitary toilets and urinals in accordance with certain embodiments of the disclosure. The controller 420 can include a processor 1405 for executing certain operational aspects associated with implementing certain systems and methods for irradiation of sanitary toilets and urinals in accordance with certain embodiments of the disclosure. The processor 1405 can be capable of communicating with a memory 1425. The processor 1405 can be implemented and operated using appropriate hardware, software, firmware, or combinations thereof. Software or firmware implementations can include computer-executable or machine-executable instructions written in any suitable programming language to perform the various functions described. In one embodiment, instructions associated with a function block language can be stored in the memory 1425 and executed by the processor 1405.

The memory 1425 can be used to store program instructions that are loadable and executable by the processor 1405 as well as to store data generated during the execution of these programs. Depending on the configuration and type of the controller 420, the memory 1425 can be volatile (such as random-access memory (RAM)) and/or non-volatile (such as read-only memory (ROM), flash memory, etc.). In some embodiments, the memory devices can also include additional removable storage 1430 and/or non-removable storage 1435 including, but not limited to, magnetic storage, optical disks, and/or tape storage. The disk drives and their associated computer-readable media can provide non-volatile storage of computer-readable instructions, data structures, program modules, and other data for the devices. In some implementations, the memory 1425 can include multiple different types of memory, such as static random-access memory (SRAM), dynamic random-access memory (DRAM), or ROM.

The memory 1425, the removable storage 1430, and the non-removable storage 1435 are all examples of computer-readable storage media. For example, computer-readable storage media can include volatile and non-volatile, removable and non-removable media implemented in any method or technology for storage of information such as computer-readable instructions, data structures, program modules or other data. Additional types of computer storage media that can be present include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile discs (DVD) or other optical storage, magnetic cassettes, magnetic tapes, magnetic disk storage or other magnetic storage devices, or any other medium which can be used to store the desired information and which can be accessed by the devices. Combinations of any of the above should also be included within the scope of computer-readable media.

Controller 420 can also include one or more communication connections 1410 that can allow a control device (not shown) to communicate with devices or equipment capable of communicating with the controller 420. The controller can also include a computer system (not shown). Connections can also be established via various data communication channels or ports, such as USB or COM ports to receive cables connecting the controller 420 to various other devices on a network. In one embodiment, the controller 420 can include Ethernet drivers that enable the controller 420 to communicate with other devices on the network. According to various embodiments, communication connections 1410 can be established via a wired and/or wireless connection on the network.

The controller 420 can also include one or more input devices 1415, such as a keyboard, mouse, pen, voice input device, gesture input device, and/or touch input device. It can further include one or more output devices 1420, such as a display, printer, and/or speakers.

In other embodiments, however, computer-readable communication media can include computer-readable instructions, program modules, or other data transmitted within a data signal, such as a carrier wave, or other transmission. As used herein, however, computer-readable storage media do not include computer-readable communication media.

Turning to the contents of the memory 1425, the memory 1425 can include, but is not limited to, an operating system (OS) 1426 and one or more application programs or services for implementing the features and aspects disclosed herein. Such applications or services can include a sensor input station 1427 for executing certain systems and methods of irradiation of sanitary toilets and urinals. The sensor input station 1427 can reside in the memory 1425 or can be independent of the controller 420. In one embodiment, the sensor input station 1427 can be implemented by software that can be provided in configurable control block language and can be stored in non-volatile memory. When executed by the processor 1405, the sensor input station 1427 can implement the various functionalities and features associated with the controller 420 described in this disclosure.

As desired, embodiments of the disclosure may include a controller 420 with more or fewer components than are illustrated in FIG. 14. Additionally, certain components of the controller 420 of FIG. 14 may be combined in various embodiments of the disclosure. The controller 420 of FIG. 14 is provided by way of example only.

References are made to block diagrams of systems, methods, apparatuses, and computer program products according to example embodiments. It will be understood that at least some of the blocks of the block diagrams, and combinations of blocks in the block diagrams, may be implemented at least partially by computer program instructions. These computer program instructions may be loaded onto a general-purpose computer, special purpose computer, special purpose hardware-based computer, or other programmable data processing apparatus to produce a machine, such that the instructions which execute on the computer or other programmable data processing apparatus create means for implementing the functionality of at least some of the blocks of the block diagrams, or combinations of blocks in the block diagrams discussed.

These computer program instructions may also be stored in a non-transitory computer-readable memory that can direct a computer or other programmable data processing apparatus to function in a particular manner, such that the instructions stored in the computer-readable memory produce an article of manufacture including instruction means that implement the function specified in the block or blocks. The computer program instructions may also be loaded onto a computer or other programmable data processing apparatus to cause a series of operational steps to be performed on the computer or other programmable apparatus to produce a computer implemented process such that the instructions that execute on the computer or other programmable apparatus provide task, acts, actions, or operations for implementing the functions specified in the block or blocks.

One or more components of the systems and one or more elements of the methods described herein may be implemented through an application program running on an operating system of a computer. They also may be practiced with other computer system configurations, including hand-held devices, multiprocessor systems, microprocessor based or programmable consumer electronics, mini-computers, mainframe computers, and the like.

Application programs that are components of the systems and methods described herein may include routines, programs, components, data structures, and so forth that implement certain abstract data types and perform certain tasks or actions. In a distributed computing environment, the application program (in whole or in part) may be located in local memory or in other storage. In addition, or alternatively, the application program (in whole or in part) may be located in remote memory or in storage to allow for circumstances where tasks may be performed by remote processing devices linked through a communications network.

### Example Process(es)

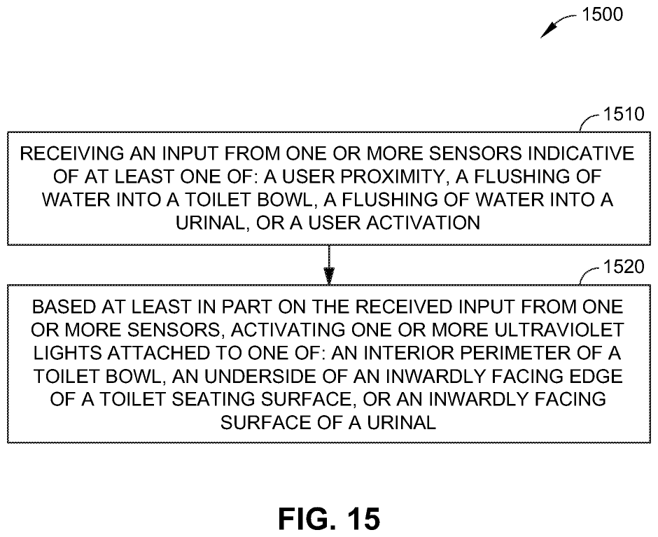

FIG. 15 illustrates an example process 1500 for irradiating a sanitary system, in accordance with an example embodiment of the disclosure. In general, operations of disclosed processes (e.g., process 1500) may be performed in an arbitrary order, unless otherwise provided in the claims. The process proceeds by receiving 1510 an input from one or more sensors that may be indicative of at least one of: a user proximity, a flushing of water into a toilet bowl, a flushing of water into a urinal, or a user activation. The process may further include activating 1520 one or more ultraviolet lights based at least in part on the received input from one or more sensors. The one or more ultraviolet lights may be attached to one of: an interior perimeter of a toilet bowl, an underside of an inwardly facing edge of a toilet seating surface, or an inwardly facing surface of a urinal.

### CONCLUSION

It should be understood that the above detailed description is to be construed as exemplary only and does not describe every possible embodiment since describing every possible embodiment would be impractical. In light of the teachings and disclosures herein, numerous alternative embodiments may be implemented.

It should be also appreciated that while the following disclosure refers to toilet bowls and other sanitary systems, embodiments of the invention may be utilized with other types of systems that may require irradiation. In some exemplary embodiments of the invention, the ultraviolet lights may be provided along a floor of a restroom in order to irradiate any spillage associated with usage of a urinal or toilet system. It should therefore be noted that throughout the description, “toilet” may be replaced by “sanitary system” or any of the above-mentioned examples; and “user” could be replaced with “operator,” “person,” or the like.

Although the technology has been described with reference to the embodiments illustrated in the attached drawing figures, equivalents may be employed, and substitutions made herein without departing from the scope of the technology as recited in the claims. For example, the components described herein need not be physically connected to one another since wireless communication among the various depicted components is permissible and intended to fall within the scope of the present invention. Components illustrated and described herein are merely examples of a device and components that may be used to implement the embodiments of the present invention and may be replaced with other devices and components without departing from the scope of the invention.

Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. An irradiation system, comprising:
one or more ultraviolet lights attached to one of: an interior perimeter of a toilet bowl, an underside of an inwardly facing edge of a toilet seating surface, or an inwardly facing surface of a urinal;
a controller connected to a power source and configured to activate the one or more ultraviolet lights;

2. The system of claim 1, wherein the one or more ultraviolet lights comprises at least one of: Ultraviolet-C lights, Ultraviolet-B lights, or Ultraviolet-A lights.

3. The system of claim 1, wherein the one or more ultraviolet lights are attached below a rim in the interior perimeter of the toilet bowl.

3. The system of claim 1, wherein the one or more ultraviolet lights at least partially define an interior perimeter of the urinal.

4. The system of claim 1, wherein the controller is configured to activate the one or more ultraviolet lights based at least in part on an input from one or more sensors indicative of at least one of: a user proximity, an impending flushing of water into the toilet bowl, a flushing of water into the urinal, or a user activation.

5. The system of claim 1, wherein the one or more ultraviolet lights is attached via one or more of: a water insoluble resin, a glass material, a poly-vinyl chloride (PVC) material or an acrylic based material.

6. The system of claim 1, wherein radiation from the one or more ultraviolet lights completely covers an area formed by one of: a toilet bowl opening or a urinal opening.

7. The system of claim 1, wherein the one or more ultraviolet lights remain activated for a period of time after the flushing of water into the toilet bowl and wherein the one or more ultraviolet lights remain activated for a period of time after the flushing of water into the urinal.

8. A method of irradiating a sanitary system, comprising:
receiving an input from one or more sensors indicative of at least one of: a user proximity, a flushing of water into a toilet bowl, a flushing of water into a urinal, or a user activation;
based at least in part on the received input from one or more sensors, activating one or more ultraviolet lights attached to one of: an interior perimeter of the toilet bowl, an underside of an inwardly facing edge of a toilet seating surface, or an inwardly facing surface of the urinal.

9. The method of claim 8, wherein the one or more ultraviolet lights comprises at least one of: Ultraviolet-C lights, Ultraviolet-B lights, or Ultraviolet-A lights.

10. The method of claim 8, wherein the one or more ultraviolet lights are attached below a rim in the interior perimeter of the toilet bowl.

11. The method of claim 8, wherein the one or more ultraviolet lights at least partially define an interior perimeter of the urinal.

12. The method of claim 8, wherein the controller is configured to activate the one or more ultraviolet lights based at least in part on an input from one or more sensors indicative of at least one of: a user proximity, an impending flushing of water into the toilet bowl, a flushing of water into the urinal, or a user activation.

13. The method of claim 8, wherein the one or more ultraviolet lights is attached via one or more of: a water insoluble resin, a glass material, a poly-vinyl chloride (PVC) material or an acrylic based material.

14. The method of claim 8, wherein radiation from the one or more ultraviolet lights completely covers an area formed by one of: a toilet bowl opening or a urinal opening.

15. A system comprising:
a processor; and
a memory comprising computer-executable instructions operable to:
receive an input from one or more sensors indicative of at least one of: a user proximity, a flushing of water into a toilet bowl, a flushing of water into a urinal, or a user activation;
based at least in part on the received input from one or more sensors, activate one or more ultraviolet lights attached to one of: an interior perimeter of the toilet bowl, an underside of an inwardly facing edge of a toilet seating surface, or an inwardly facing surface of the urinal.

16. The system of claim 15, wherein the one or more ultraviolet lights comprises at least one of: Ultraviolet-C lights, Ultraviolet-B lights, or Ultraviolet-A lights.

17. The system of claim 15, wherein the one or more ultraviolet lights are attached below a rim in the interior perimeter of the toilet bowl.

18. The system of claim 15, wherein the one or more ultraviolet lights at least partially define an interior perimeter of the urinal.

19. The system of claim 15, activating the one or more ultraviolet lights is based at least in part on an input from one or more sensors indicative of at least one of: a user proximity, a flushing of water into the toilet bowl, an impending flushing of water into the urinal, or a user activation.

20. The system of claim 1, wherein the one or more ultraviolet lights is attached via one or more of: a water insoluble resin, a glass material, a poly-vinyl chloride (PVC) material or an acrylic based material.
